(19) United States
(12) Patent Application Publication
(10) Pub. No.: US 2020/0208291 A1
(43) Pub. Date: Jul. 2, 2020

(54) NANOSTRUCTURED MAGNESIUM MATERIALS, METHODS AND DEVICES

(71) Applicants: Luisa Fernanda BERRIO, Medellin (CO); Felix ECHEVERRIA, Medellin (CO); The Board of Trustees of the University of Illinois, Urbana, IL (US)

(72) Inventors: Jean Paul ALLAIN, Champaign, IL (US); Akshath SHETTY, Mumbai (IN); Luisa Fernanda BERRIO, Medellin (CO); Felix ECHEVERRIA, Medellin (CO)

(73) Assignee: The Board of Trustees of the University of Illinois, Urbana, IL (US)

(21) Appl. No.: 16/500,685

(22) PCT Filed: Apr. 6, 2018
(86) PCT No.: PCT/US18/26582

§ 371 Date: Oct. 3, 2019

Related U.S. Application Data
(60) Provisional application No. 62/483,105, filed on Apr. 7, 2017.
(60) Provisional application No. 62/483,074, filed on Apr. 7, 2017.
(60) Provisional application No. 62/556,048, filed on Sep. 8, 2017.
(60) Provisional application No. 62/556,120, filed on Sep. 8, 2017.

(51) Int. Cl. C25D 11/30; C25D 11/02; A61L 27/04; A61L 27/56

(52) U.S. Cl. CPC C25D 11/30; C25D 11/022; B82Y 40/00; A61L 27/56; A61L 27/047

(57) ABSTRACT

Provided herein are methods for the controlled, independent modification of the surface of magnesium-based materials and compositions generated thereby. The methods allow for the alteration of multiple surface characteristics including generation of precise nanostructures, morphology, crystallography, chemical hybridizations and chemical composition for controlled bioresorption and/or increased biocompatibility, for example, osseointegration, hydroxyapatite formation, osseoconduction, cell adhesion, cell proliferation, enhanced local mechanical properties (elasticity, modulus, surface texture, porosity), hydrophobicity, hydrophilicity, steric hindrance, modulating-immuno response, anti-inflammatory properties and/or anti-bacterial properties.

### CROSS-REFERENCE TO RELATED APPLICATIONS

This application claims the benefit of and priority to U.S. Provisional Application No. 62/483,105, filed Apr. 7, 2017; U.S. Provisional Application No. 62/483,074, filed Apr. 7, 2017; US Provisional Application No. 62/556,120, filed Sep. 8, 2017 and US Provisional Application No. 62/556,048, filed Sep. 8, 2017, which are each hereby incorporated by reference in their entirety to the extent not inconsistent herewith.

### BACKGROUND OF INVENTION

Magnesium and magnesium alloys are widely recognized as advantageous materials for biological implants, including bone implants, tissue scaffolds and venous implants. Magnesium is typically preferred for applications in which the implant is temporary, as magnesium naturally resorbs into solution when contacted with a biological fluid and magnesium is common in the human body. Thus, magnesium implants safely resorb over a period of time and do not require surgical removal after the implant has served its purpose. Additionally, magnesium is attractive as an implant material for its mechanical properties. For example, implants may be tailored to closely mimic the tissue in which the implant is interacting, such as bone, providing more successful implantation, faster healing and increased integration between the implant and the host tissue.

A major drawback of magnesium implants is that the bioresorption of the implant occurs heterogeneously and rapidly, which can compromise the mechanical strength of the implant. Additionally, the bioresorption process may generate hydrogen gas. While small amounts of hydrogen gas can be naturally removed by the body, larger quantities generated by rapid bioresorption can lead to inflammation and necrosis.

Modern implant producers have attempted to address these issues in a number of ways. Commonly, magnesium implants are surface treated, such as by anodization, to reduce the rate at which the implant resorbs when in contact with at biological fluid. One method of surface treatment is chemically based. The implant is treated by using one or more chemicals which alter the composition of the surface layer, resulting in a lower rate of resorption and a higher implant lifetime. However, these chemical treatments often use expensive, dangerous and toxic chemicals, increasing both the cost of the implant as well as the cost of properly disposing of byproducts. Further, chemical treatments are limited in that they typically provide a heterogeneous layer across the implant and lack precise control over bioresorption rates.

Plasma has also been used to alter chemical and mechanical properties of magnesium implants. However, known methods of plasma treatment (e.g. kinetic roughening) are imprecise and provide little control over the plasma surface interaction. By providing precise porous and/or nanopatterned regions to the implant, including tailored to the specific application of the implant, bioresorption and biomechanical stresses may be reduced and cell adhesion increased, resulting in longer implant life, faster patient recovery times and reduced risk of implant tissue damage, complications and infections.

Nanopatterned surfaces have been obtained mostly by bottom-up and top-down techniques on model materials given the difficulty in high-fidelity control of clinically-relevant surfaces and of complex 3D systems. Furthermore, no current nanoscale modification method exists that can control both surface chemistry and topography independently.

Thus, it can be seen from the foregoing that there remains a need in the art for surface modification of magnesium-based implants, including at the nanometer level to improve bioresorption rates, biomechanical compatibility, associated functional deployment and implant lifetime and success rate, including by well controlled cellular adhesion to implant surface, morphology and behavior as well as high-fidelity control of the spatio-temporal immune-response behavior of the biomedical implant interface with the body. Tunability of the implant surface to engender specific bioresorption profiles and mechanical properties is desirable.

### SUMMARY OF THE INVENTION

Provided herein are methods for the controlled, independent modification of the surface of magnesium-based materials and compositions generated thereby. The methods allow for the alteration of multiple surface characteristics including generation of precise nanostructures, morphology, crystallography, chemical hybridizations and chemical composition for controlled bioresorption and/or increased biocompatibility, for example, osseointegration, hydroxyapatite formation, osseoconduction, cell adhesion, cell proliferation, enhanced local mechanical properties (elasticity, modulus, surface texture, porosity), hydrophobicity, hydrophilicity, steric hindrance, modulating-immuno response, anti-inflammatory properties and/or anti-bacterial properties. The surface of the composition may be modified by independently controlling parameters (e.g. incident angle, fluence, flux, energy, species, etc.) of one or more directed energetic particle beams, providing more control and increased bioactivity over conventional kinetic roughing techniques.

The provided compositions are modified to provide controlled bioresorption profiles and/or alter biological properties or functions. The provided methods are precise, allowing for the controlled generation of specific nanostructures across multiple domains. Further, precise changes to crystallography or morphology are possible, including changes to grain structure and the generation of metastable states. The provided methods also allow for specific modification of chemical composition, for instance, accurate creation of one or more alloys different from adjacent domains or the original underlying substrate, including the generation of aluminum oxide layers to promote hydroxyapatite formation. Irradiation-driven compositional variation such as one element over another at the surface differing from the sub-surface can be tuned to specific concentrations.

In aspect, provided is a magnesium composition comprising: a magnesium containing substrate having a surface; wherein the surface has a plurality of nanoscale domains characterized by a surface geometry providing a selected multifunctional bioactivity; wherein each of the nanoscale domains has at least one lateral spatial dimension selected over the range of 3 nm to 1 μm and a vertical spatial dimension less than 500 nm.

In an aspect, provided is magnesium composition comprising: a magnesium containing substrate having a surface; wherein the surface has a plurality of nanoscale domains characterized by a surface geometry providing a selected multifunctional bioactivity; wherein the nanoscale domains are generated by exposing the surface to one or more directed energetic particle beam characterized by one or more beam properties.

In an embodiment, for example the magnesium containing substrate is a magnesium metal, a magnesium alloy, an anodized magnesium metal, an anodized magnesium alloy or a magnesium oxide. In an embodiment, the selected multifunctional bioactivity is with respect to an in vivo or in vitro activity relative to an unmodified magnesium containing substrate, for example, a magnesium containing substrate surface not having the plurality of nanoscale domains characterized by the nanofeatured surface geometry.

The provided compositions and methods may provide controlled bioresorption, increasing potential effectiveness as biological implants. The control of bioresorption may make magnesium preferable to other implants as it can be safely resorbed into the body as elemental magnesium. Further, control of bioresorption allows for the mechanical properties of the implant or device to change over time. Additionally, bioresorption of magnesium may generate hydrogen, which can be controlled by controlling the rate of bioresorption.

The provided compositions and methods may utilize anodized magnesium. Nanostructured anodized magnesium may be useful, for example, for bone implants in which the magnesium composition mimics the structural and mechanical properties of bone. Surface modification may allow for the promotion of hydroxyapatite formation when the composition is exposed to biological fluid.

In embodiments, the in vivo or in vitro activity is an enhancement in bioresorption, hydrogen generation, cell adhesion activity, cell shape activity, cell proliferation activity, cell migration activity, cell differentiation activity, anti-bacterial activity, bactericidal activity, anti-inflammatory activity, osseointegration activity, biocorrosion activity, cell differentiation activity, immuno-modulating activity during acute or chronic inflammation or any combination of these. In an embodiment, the enhancement of in vivo or in vitro activity is equal to or greater than 50%, equal to or greater than 100%, or optionally, equal to or greater than 150%.

In an embodiment, the magnesium containing substrate is anodized. In embodiments, the in vivo or in vitro activity is a change in rate of bioresorption. In embodiments, the change in rate of bioresorption is selected from the range of 0.10 mm/year to 0.18 mm/year. In embodiments, the in vivo or in vitro activity is a decrease in hydrogen generation, for example, a decrease in hydrogen generation greater than or equal to 0.6 ml/cm².

In an embodiment, for example, the nanoscale domains comprise calcium phosphate. In an embodiment, the nanoscale domains comprise an increase in Al₂O₃ content relative to the Al₂O₃ content of other regions of the magnesium containing substrate no having the nanoscale domains.

The described methods and compositions may include surface geometries and nanoscale domains which incorporate nanostructures as well as changes in composition, surface charge density and crystallography. Advantageously, these properties may be independently tuned by varying beam properties, allowing for enhancement of multiple biological properties and responses.

In an embodiment, the surface geometry is spatial distribution of relief features, recessed features, localized regions characterized by a selected composition, phase, crystallographic texture, or any combination of these. In embodiments, for example, the surface geometry is a periodic or semi-periodic spatial distribution of the nanoscale domains. In embodiments, the surface geometry is provided between and within pores of the substrate. In embodiments, the surface geometry is a selected topology, topography, morphology, texture or any combination of these.

In an embodiment, for example, each of the nanoscale domains are characterized by a vertical spatial dimension of less than or equal to 50 nm, less than or equal to 25 nm, or optionally, less than or equal to 10 nm. In embodiments, each of the nanoscale domains are characterized by a vertical spatial dimension selected over the range of 10 nm to 250 nm. In embodiments, the nanoscale domains comprise nanowalls, nanorods, nanoplates, nanoripples or any combination thereof having lateral spatial dimensions selected over the range of 10 to 1000 nm and vertical spatial dimensions of less than or equal to 250 nm.

In embodiments, the nanowalls, nanorods, nanoplates or nanoripples are inclined towards a direction oriented along a selected axis relative to the surface. In embodiments, for example, the nanowalls, nanorods, nanoplates or nanoripples are separated from one another by a distance of less than or equal to 200 nm, less than or equal to 100 nm, or optionally, less than or equal to 50 nm.

In embodiments, the nanoscale domains comprise discrete crystallographic domains. In embodiments, the nanoscale domains characterized by a chemical composition different from the bulk phase of the magnesium containing substrate.

In an embodiment, the surface geometry provides an enhancement in vivo or in vitro activity with respect to cell adhesion proliferation activity and migration greater than or equal to 100%. In embodiments, the surface geometry provides an enhancement in vivo or in vitro activity with respect to anti-bacterial activity and bactericidal activity greater than or equal to 100%. In embodiments, for example, the surface geometry provides a local in vivo increase in pH, wherein the pH is increased by 0.25 or more, 0.5 or more, or optionally, 1.0 or more.

In embodiments, the surface geometry provides an enhancement of a selected physical property of the substrate, for example, hydrophilicity, hydrophobicity, surface free energy, surface charge density or any combination of these. In embodiments, the enhancement of selected physical property is equal to or greater than 25%, equal to or greater than 50%, or optionally, equal to or greater than 75%.

In embodiments, the magnesium containing substrate is a biocompatible substrate. In embodiments, the magnesium containing comprises a mesoporous, microporous, or a nanoporous substrate. In an embodiment, the magnesium containing substrate comprises a component of a medical device.

In embodiments, the directed energetic particle beam is a broad beam, focused beam, asymmetric beam, reactive beam or any combination of these. In embodiments, the one or more beam properties is intensity, fluence, energy, flux, incident angle, ion composition, neutral composition, ion to neutral ratio or any combinations thereof.

In an aspect, provided is a method of fabricating a bioactive magnesium substrate comprising: a) providing the magnesium containing substrate having a substrate surface; and b) directing a directed energetic particle beam onto the substrate surface, thereby generating a plurality of nanoscale domains on the surface; wherein the directed energetic particle beam has one or more beam properties selected to generate the plurality of nanoscale domains characterized by a surface geometry providing a selected multifunctional bioactivity.

In an embodiment, for example, the directed energetic particle beam is a broad beam, focused beam asymmetric beam or any combination of these. In embodiments, the directed energetic particle beam onto the substrate surface comprises directed irradiation synethesis (DIS), directed plasma nanosynthesis (DPNS), Direct Seeded Plasma Nanosynthesis (DSDPNS), Direct Soft Plasma nanosynthesis (DSPNS) or any combination of these.

In an embodiment, for example, the multifunctional bioactivity comprises bioresorption. In an embodiment for certain applications, the step of directing the directed energetic particle beam onto the substrate surface is achieved using a method other than directed irradiation synthesis (DIS). For example, the invention, includes methods of fabricating a bioactive magnesium containing substrate wherein directed plasma nanosynthesis (DPNS), direct seeded plasma nanosynthesis (DSPNS) or any combination of these techniques is used to carry out the step of directing the directed energetic particle beam onto the substrate surface to generate a plurality of nanoscale domains characterized by a surface geometry providing a selected multifunctional bioactivity. Accordingly, one of skill in the art will readily understand that certain applications and materials of the invention are achieved using methods that do not include processing via directed irradiation synthesis (DIS).

In embodiments, the one or more beam properties is intensity, fluence, energy, flux, incident angle, ion composition, neutral composition ion to neutral ratio or any combinations thereof. In embodiments, the directed energetic particle beam comprises one or more ions, neutrals or combinations thereof. In an embodiment, for example, the ions are Ne ions, Kr ions, Ar ions, Xe ions, N ions or a combination thereof. In an embodiment, the directed energetic particle beam is generated from an energetic 02 precursor.

In embodiments, the directed energetic particle beam anodizes the magnesium containing substrate thereby generating an anodized bioactive magnesium substrate. In embodiments, the one or more beam properties comprise incident angle and the incident angle is selected from the range of 0° to 80° or selected from the range of 0° to 60°. In embodiments, the one or more beam properties comprise fluence and the fluence is selected from the range of 1×10¹⁶ cm⁻² to 1×10¹⁹ cm⁻², or optionally, 1×10¹⁶ cm⁻² to 1×10²⁰ cm⁻². In embodiments, the one or more beam properties comprise energy and the energy is selected from the range of 0.05 keV to 10 keV, or optionally, 0.1 keV to 10 keV.

In an aspect, provided is a method of fabricating a bioactive magnesium substrate comprising: a) providing the magnesium containing substrate having a substrate surface; and b) directing a first directed energetic particle beam and a second directed energy particle beam onto the substrate surface, thereby generating a plurality of nanoscale domains on the surface; wherein the first directed energetic particle beam has one or more first beam properties and the second directed energetic particle beam has one or more second beam properties; and wherein at least one of the first beam properties is different than at least one of the second beam properties and the first beam properties and the second beam properties are independently selected to generate the plurality of nanoscale domains characterized by a surface geometry providing a selected multifunctional bioactivity.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1. SEM images of conventional Mg-based material (left) and DPNS-enhanced nanostructured magnesium-based surface (right).

FIG. 2. SEM images (low and high magnification) showing the evolution of surface nano patterning of anodized magnesium phosphate and amine base compound samples for different incidence angles with oxygen irradiation.

FIG. 3. SEM images (low and high magnification showing the evolution of surface nanopatterning of anodized magnesium phosphate and pyrophosphate samples for different incidence angles with oxygen irradiation.

FIG. 4. SEM images (low and high magnification showing the evolution of surface nanopatterning of anodized magnesium phosphate and tartrate samples for different incidence angles with oxygen irradiation.

FIG. 5. SEM images (low and high magnification showing the evolution of surface nanopatterning of anodized magnesium phosphate and fluoride at constant voltage samples for different incidence angles with oxygen irradiation.

FIG. 6. SEM images (low and high magnification showing the evolution of surface nanopatterning of anodized magnesium phosphate and fluoride at constant current samples for different incidence angles with oxygen irradiation.

FIG. 7. SEM images (low and high magnification) showing the evolution of surface nano patterning of anodized magnesium phosphate and amine base compound samples for different incidence angles with argon irradiation.

FIG. 8. SEM images (low and high magnification) showing the evolution of surface nano-patterning of anodized magnesium phosphate and pyrophosphate samples for different incidence angles with argon irradiation

FIG. 9. SEM images (low and high magnification) showing the evolution of surface nano-patterning of anodized magnesium phosphate and tartrate samples for different incidence angles with argon irradiation

FIG. 10 SEM images (low and high magnification) showing the evolution of surface nano-patterning of anodized magnesium phosphate and fluoride at constant voltage samples for different incidence angles with argon irradiation.

FIG. 11. SEM images (low and high magnification) showing the evolution of surface nano-patterning of anodized magnesium phosphate and fluoride at constant current samples for different incidence angles with argon irradiation.

FIG. 12. SEM images of the five different surface modification by anodization.

FIG. 13. Osteoblast study on magnesium phosphate and amine base compound sample.

FIG. 14. Osteoblast study on magnesium phosphate and pyrophosphate sample.

FIG. 15. Osteoblast study on magnesium phosphate and tartrate sample.

FIG. 16. Osteoblast study on magnesium phosphate and fluoride at constant voltage sample.

FIG. 17. Osteoblast study on magnesium phosphate and fluoride at constant current sample.

FIG. 18. provides an EDS analysis summary of sample PA_Mg_001.

FIG. 19. provides an EDS analysis summary of sample PA_Mg_002.

FIG. 20 provides XRD data of a magnesium alloy treated with DPNS at different fluences.

FIG. 21 provides XRD data of a magnesium alloy treated with DPNS at different fluences.

FIG. 22 provides concentration data of a magnesium alloy treated with DPNS based on the data in FIGS. 21 and 22.

### DETAILED DESCRIPTION OF THE INVENTION

In general, the terms and phrases used herein have their art-recognized meaning, which can be found by reference to standard texts, journal references and contexts known to those skilled in the art. The following definitions are provided to clarify their specific use in the context of the invention.

“Nanoscale domains,” as used herein, refers to features characterized by one or more structural, composition and/or phase properties having relatively small dimensions generated on the surface of a substrate. Nanoscale domains may refer to relief features and/or recessed features such as trenches, nanowalls, nanocones, nanoplates, nanocolumns, nanoripples, nanopillars, nanorods, nanowires, nanotubes and/or quantum dots. Nanoscale domains may refer to discrete crystalline domains, compositional domains, distributions of defects, and/or changes in bond hybridization. Nanoscale domains include self-assembled nanostructures. In embodiments, for example, nanoscale domains refer to surface depths or structures generated on a surface having dimensions of less than 1 μm, less than or 500 nm, less than 100 nanometers, or in some embodiments, less than 50 nm. In an embodiment, nanoscale domains refer to a domain in a thermally stable metastate.

“Surface geometry” refers to a plurality nanoscale domains positioned on the surface of a substrate. In embodiments, for example, nanofeatured surface geometry is a periodic or semi-periodic spatial distribution of nanoscale domains. For example, nanofeatured surface geometries include topology, topography, spatial distribution of compositions, spatial distribution of phases, spatial distribution of crystallographic orientations and/or spatial distribution of defects. Surface geometries of some aspects are useful for providing a selected multifunctional bioactivity, a selected physical property or a combination thereof.

“Selected multifunctional bioactivity” refers to an enhancement of in vivo or in vitro activity with respect to a plurality of biological or physical processes. In embodiments, for example, multifunctional bioactivity is enhanced relative to a titanium or titanium alloy substrate surface not having said plurality of nanoscale domains characterized by nanoscale surface geometry. In an embodiment, for example, a selected multifunctional bioactivity is an enhancement in bioresorption, hydrogen generation, cell adhesion activity, cell shape activity, cell proliferation activity, cell migration activity, cell differentiation activity, anti-bacterial activity, bactericidal activity, anti-inflammatory activity, osseoconductive activity, osseointegration activity, biocorrosion activity, cell differentiation activity, immuno-modulating activity during acute or chronic inflammation or any combination of these. In an embodiment, for example, a selected multifunctional bioactivity is a modulation in the immune response to a foreign body (e.g. the implant). In an embodiment, for example, a selected multifunctional bioactivity is an enhancement or inhibition of one or more protein interactions.

“Directed energetic particle beam,” as used herein, refers to a stream of accelerated particles. In embodiments, the directed energetic particle beam is generated from low-energy plasma. In some embodiments, directed energetic particle beam is a focused or broad ion beams capable of delivering a controlled number of ions to a precise point or area upon a substrate over a specified time. Directed energetic particle beam may include ions and additional non-ionic particles including subatomic particles or neutral atoms or molecules. In embodiments, directed energetic particle beams provide individual ions to the target location. Examples of directed energetic particle beams include focused ion beams, broad ion beams, thermal beams, plasma generated beams and optical beams.

“Beam property” or “beam parameter” refer to a user or computer controlled property of beam, for example, an ion beam. Beam parameter may refer to incident angle with a target substrate, fluence, energy, flux, beam composition and ion species. Beam parameters may be adjusted to provide selected interactions between the beam and the target substrate to generate specific nanostructures or enhance specific properties of the substrate including rate of bioresorption. Beam parameters may be controlled by a variety of means, including adjustments to electromagnetic devices in communication with the beam, adjusting the gas or energy source used to generate the beam or physical positioning of the beam in reference to the target.

“Vertical spatial dimension” refers to a measure of the physical dimensions of a nanoscale domain perpendicular or substantially perpendicular to the planar or contoured surface of a substrate. In embodiments, vertical spatial dimension refers to a height or depth of a nanoscale domain or the mean depth of a surface modification, for example, a crystalline or compositional domain.

“Lateral spatial dimension” refers to a measure of the physical dimensions of a nanoscale domain parallel or substantially parallel to the planar or contoured surface of a substrate.

“Magnesium or Magnesium alloy substrate” refers to any substrate composed of magnesium including specific magnesium alloys described herein. In some embodiments, magnesium alloy may refer to alloys containing magnesium but in which magnesium is not the primary component. In other embodiments, magnesium alloy refers to alloys in which magnesium represents more than 25%, or optionally 50%, of the alloy. Magnesium and magnesium alloys may include an oxide layer, for example magnesium oxide or aluminum oxide, including on the surface being modified.

“Porosity” or “porous magnesium” refers to substrates or magnesium surfaces having individual or networked voids at or near the surface of the substrate. Porosity may be nanoscale, microscale or larger. As described herein, substrates may have porosity prior to any plasma treatment (e.g. porosity formed during substrate formation such as sintering). In some embodiments, pores may be formed, enlarged or altered by the treatment of directed plasma, including forming nanopatterns on interior pore surfaces or walls between individual pores.

“Multiplexing” refers to simultaneously modifying the target substrate in more than one way, for example, by providing two or more directed particle beams at the substrate having different properties, for example, to generate or modify at least one nanoscale domain (e.g. nanoscale features, crystalline domains, compositional domains, distributions of defects, changes in bond hybridization. In some embodiments, for example, a single directed particle beam may have one or more beam properties to generate or modify multiple nanoscale domains on the substrate. In embodiments, multiple direction particle beams are generated from the same plasma source.

The technology as described in the present disclosure includes an advanced nanomanufacturing process as described herein, advanced tools particular for this process and a number of unique nano-scale structures generated as a result of the processing.

In one embodiment, provided is an atomic-scale additive nanomanufacturing process capable of transforming materials with multi-functional properties without the need for expensive heat cycles, toxic chemical processes or thermodynamic limitations of material compatibility in processing. The interface between plasma and material becomes an open thermodynamic system driven far from equilibrium by a rich variety of physical mechanisms, including high-energy kinetic disordering, compositional phase dynamics, and the emergence of metastable material states. The instabilities that arise due to these mechanisms lead to the evolution of well-ordered nanostructures, the compositional and morphological characteristics of which dictate the material properties.

“Directed energetic particle beams” are drawn from a low-temperature plasma (gas discharge) in a manner that controls the energy, species and intensity of the respective beams from the aforementioned plasma. This technique may be called directed plasma nanosynthesis (DPNS) herein. The particles may be combined with additional reactive atoms and/or surfactants that interact with material surface inducing variation in a number of properties including: surface chemistry, composition, topography, topology, charge density and bond hybridization. In some cases the technology can manipulate these properties independently providing for multi-functionality on the material surface without modification to the bulk material. Depending on material type the energetic particles are selected both in mass and species to result in the desired material property (e.g. hydrophobicity, anti-bacterial for biomaterials, etc. . . . ). The material can be a polymer, metal, ceramic, or semiconductor and the synthesis can be done over large areas, at room temperature and over a short period of time (e.g. seconds). DPNS is designed to independently modify surface topography, composition and charge density yielding increase of surface energy and surface-to-volume ratios by factors of 50-100% and 100-1000, respectively. DPNS include a use of a plasma source enabling the modification of existing product materials (e.g. on a biomedical stent, implant device, etc. . . . ) improving their properties or synthesizing completely new class of materials. DPNS enables a single source that addresses the problematic use of thin-film coatings for bioactive interfaces, which can potentially lead to osteolysis and chronic inflammation. Coating disintegration and delamination is also a prevalent problem that cannot be solved with current synthesis approaches that include: electrophoretic deposition, anodization, electrolysis, reactive DC magnetron sputtering, RF plasma sputtering, and x-ray sintering among others. One of the primary issues with these conventional technologies is the formation of the interface between the coating and biomedical material substrate. Therefore, features of DPNS are: 1) low cost (e.g. they are a low-temperature process; heat cycles during synthesis make-up 30-40% of the current processing cost of surface modification techniques), 2) green and sustainable (does not require harsh chemicals for synthesis and can enhance usability of natural materials), and 3) scalable (particle irradiation can be conducted throughput levels of about 1012 micron2/hr or a modification of a 6-inch wafer in about 10 seconds). Another added benefit and potentially disruptive approach is the ability to modify a surface composition and chemistry independent of the topography with high-fidelity. In other words, inducing a surface that can potentially enhance cell adherence and proliferation while repelling bacteria, for example.

“Directed energetic particle beams” include DPNS to produce nanostructures on the substrate surface. In first step, a substrate is provided in a fixture, not shown, where the directed energetic particle beam from a low temperature plasma may operate on the substrate with a surface. The directed energetic particle beam(s) from a low temperature plasma source are directed to the substrate surface in accordance with parameters and/or properties that correspond to a desired nanostructure topology. The parameter control may occur in an automated fashion, such as under the control of a numerical control device or special purpose computer, including a processing device and a memory containing programming instructions (not shown). In an optional step, additional beam(s) may be generated and directed to the surface of the substrate also in accordance with parameters and/or properties that correspond to a desired nanostructure topology. Optional step includes depositing one or more agents on the surface of the substrate.

“Directed energetic particle beams” can be derived from plasma processing sources known in the art, for example, Tectra GmbH Physikalische Instrumente (GENII PLASMA ION SOURCE) and Oxford Instruments (ISE 5 ion sputtering source). Also SVT Associates, Inc. provides the RF-6.02 Plasma Source. While the principles and methods for creating plasma sources are known, these plasma processing methods create only mono-directional particle beams, which limits their usage to flat, 2D surfaces. Methods for performing DPNS as 3D are described in, for example, U.S. Patent Application Ser. No. 62/483,105, “Directed Plasma Nanosynthesis (DPNS) Methods, Uses and Systems,” filed Apr. 7, 2017, the disclosure of which is incorporated by reference herein in its entirety.

“Directed energetic particle beams” include low temperature plasmas and gasiform plasmas with electron temperature under 10 eV, electron density typically from 1014 to 1024 m⁻³. In general, low temperature plasmas have a low degree of ionization at low densities. This means the number of ions and electrons is much lower than the number of neutral particles (molecules). Different particles inside the plasma, i.e. neutrals, ions and electrons, can have different temperatures or energies. Indeed, in many applications, the background gas is near room temperature. In this regard, gas phase reaction activation energy can be driven by electron impact rather than thermally and the substrate is not subjected to extreme heating, which is useful for functionalizing temperature sensitive substrates such as polymers. For “directed energetic particle beams” one or more beam properties is the gas, intensity, fluence, energy, flux, incident angle, species mass, charge, cluster size, molecule or any combinations thereof. In an embodiment, for example, the directed energetic particle beam comprises one or more ions, neutrals or combinations thereof. In embodiments, the one or more beam properties are the ion composition, neutral composition, the ratio of ion abundance to neutral abundance or any combination of these. In embodiments, the directed energetic particle beam is incident upon the substrate from a plurality of directions.

Using “directed energetic particle beams” nanostructures may be obtained as function of energetic particle species, fluence and incident angle with respect to the surface normal. For example, energetic particle species may include those obtained from gases such as Kr, Ar, Ne, Xe, H, He, O2 and/or N2. Fluence can be, for example, between 1×10¹⁷ to 1×10¹⁸ particles per second per square meter, but may vary from 0.1×10¹⁷ to 50×10¹⁷. In some embodiments, fluence is 1×10¹⁷, 2.5×10¹⁷, 5×10¹⁷, or 1×10¹⁸ particles per second per square meter. Finally, incident angle may be varied in single degrees between the angles of 0 and 80 degrees, in some embodiments, for example, 30 degrees, 60 degrees, and 80 degrees. In some embodiments, for example, the plasma-based source of the invention provides one or more directed particle beams having a distribution of incident angles, such as a distribution of incident angles characterized between 0 and 90 degrees with respect to the sample surface normal.

The invention may be further understood by reference to the following non-limiting Examples that expand on certain aspects and embodiments of the invention.

### Example 1—Nanostructured Bioactive Anodized Magnesium-Based Materials

This example describes combining anodized porous magnesium exposed to directed plasma nanosynthesis (DPNS) in order to improve biocompatibility, control corrosion and transforms the surface to hydroxyapatite phases depending on the particular anodized coating chemistry with the potential to enhance osseointegration and osseoconduction.

Metallic and ceramic materials are commonly used as bone implants, however many materials exhibit large differences in mechanical behavior when integrated with bone. When there is a large difference in the mechanical properties of the implant and the host bone tissue there is the possibility of implant rejection, for example, due to a process known as stress shielding. Improving the osseointegration between bone tissue and implant may provide a better healing response. Magnesium has received increased attention as a biomaterial due to its attractive biomechanical properties that may mimic the strength of bone much more closely than other candidate metallic implant materials. Furthermore, magnesium is an abundant metal in the human body and therefore is assimilated naturally makes magnesium a good candidate as a bioabsorbable, temporary implant that can be designed to gradually decrease in strength as the host bone tissue increases its bearing load [P. K. Chu 2015, J. Mater. Chem. B 2015, 3, 2024-2042].

However, magnesium-based implants also have limitation. Currently, magnesium implants are restricted by the tendency of this material to resorb in a heterogeneously way and at high speed when it is in contact with body fluid which compromises the mechanical performance of the implant. The rapid dissolution of Mg generates hydrogen gas in amounts that the human body cannot absorb in a proper way, which promotes the appearance of skin inflammation due to the hydrogen bubble effect around the implant inducing necrosis. Additionally, the presence of large amounts of hydrogen increases the pH level, altering the area adjacent to the biological environment and generating medical complications that result in longer treatment times, decreased quality of life of patients and additional treatment and patient care costs.

Improvement and control of corrosion resistance of magnesium materials can be achieved via anodization, namely using plasma electrolytic oxidation. With this approach, the metal acts as the anode and is converted to an oxide film having desirable corrosion protective, decorative, and functional properties. Anodization can increase the film thickness, hardness, corrosion resistance, and wear resistance and provide better adhesion for primers than the bare metal. The anodizing behavior of magnesium materials is strongly influenced by the voltage or current applied. Different passive and active states can be found depending on the applied voltage/current, time, substrate, and electrolyte. However, when anodization is used on materials to be applied in a bioenvironment, there are certain requirements for the coating. Typically, the coating is designed to be thick, strong, and nontoxic requiring additional anodization constraints.

Described herein is surface modification of anodized magnesium materials using DPNS. In addition to modifying the surface of the magnesium to control corrosion rate, changes may be induced on the anodized surface such as surface tension, hydrophilicity and bioactivity in order to improve its performance as a bone implant material. DPNS uses irradiation by ions and neutrals extracted from a plasma at specific parameters, for example, effluence and energy, enabling the modification of surface energy, surface topography and a number of additional properties rendering magnesium-based materials bioactive and biocompatible. Additionally, DPNS allow for modification of magnesium surfaces to promote hydroxyapatite phase formations and/or cell growth in addition to the control of corrosion.

DPNS generates patterned structures and unique topography at the nanoscale while eliminating the shortcomings of chemical approaches that when scaled to industrial levels increase the production of toxic chemical waste and ultimately processing cost. DPNS is a physical modification approach and inherently scalable by virtue of its intrinsic large-area simultaneous exposure of materials surfaces and interfaces. Thus DPNS may provide high-value and high-volume manufacturing.

One feature of DPNS is the formation of surface nanostructures on porous anodized Mg-based materials for the application as a biomaterial for enhanced osseoconduction and osseointegration. The result is enhanced bone cell integration by transformation of magnesium to HA phases when exposed to body fluid, corrosion control and biomechanical strength control. In particular, this has important ramifications for design pathways focusing on tuning bioactive properties used in multiple applications potentially increasing biocompatibility and biosurface material adaptability.

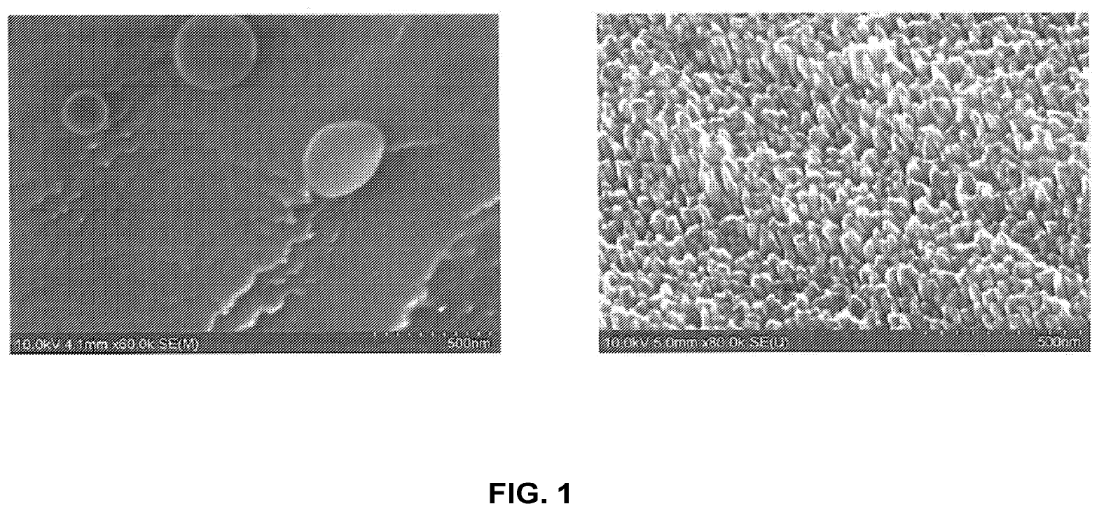

FIG. 1 shows an example of a standard magnesium surface with its intrinsic surface topography (left) used in current conventional magnesium-based biomaterials. On the right, the DPNS-modified surface. The micrographs are shown at similar magnification.

Several example coatings have been designed to control corrosion properties may be introduced via anodization using plasma electrolytic oxidation:

1) Phosphate and amine based compound (Mg_PA)
2) Phosphate and fluoride at constant voltage (Mg_PFCV)
3) Phosphate and fluoride at constant current (Mg_PFCC)

### 4) Phosphate and Pyrophosphate (Mg_PP)

### 5) Phosphate and Tartrate (Mg_PT)

DPNS conditions of different anodized magnesium samples are summarized in Table 1 and Table 2. Oxygen (O₂) and Argon (Ar) sources at 1 keV were used to irradiate Mg samples; 0° and 60° were the incidence angles.

TABLE 1
Irradiation Parameters (1-keV O₂) on anodized magnesium samples

| Sample | Gas | Energy (eV) | Angle (degree) | Fluence (cm⁻²) |
|---|---|---|---|---|
| Mg_PA_001 | O2 | 1000 | 0 | 1.00E+18 |
| Mg_PA_002 | O2 | 1000 | 60 | 1.00E+18 |
| Mg_PP_001 | O2 | 1000 | 0 | 1.00E+18 |
| Mg_PP_002 | O2 | 1000 | 60 | 1.00E+18 |
| Mg_PT_001 | O2 | 1000 | 0 | 1.00E+18 |
| Mg_PT_002 | O2 | 1000 | 60 | 1.00E+18 |
| Mg_PFCV_001 | O2 | 1000 | 0 | 1.00E+18 |
| Mg_PFCV_002 | O2 | 1000 | 60 | 1.00E+18 |
| Mg_PFCC_001 | O2 | 1000 | 0 | 1.00E+18 |
| Mg_PFCC_002 | O2 | 1000 | 60 | 1.00E+18 |

TABLE 2
Irradiation Parameters (1-keV Ar) on anodized magnesium samples

| Sample | Gas | Energy (eV) | Angle (degree) | Fluence (cm⁻²) |
|---|---|---|---|---|
| Mg_PA_003 | Ar | 1000 | 0 | 1.00E+18 |
| Mg_PA_004 | Ar | 1000 | 60 | 1.00E+18 |
| Mg_PP_003 | Ar | 1000 | 0 | 1.00E+18 |
| Mg_PP_004 | Ar | 1000 | 60 | 1.00E+18 |
| Mg_PT_003 | Ar | 1000 | 0 | 1.00E+18 |
| Mg_PT_004 | Ar | 1000 | 60 | 1.00E+18 |
| Mg_PFCV_003 | Ar | 1000 | 0 | 1.00E+18 |
| Mg_PFCV_004 | Ar | 1000 | 60 | 1.00E+18 |
| Mg_PFCC_003 | Ar | 1000 | 0 | 1.00E+18 |
| Mg_PFCC_004 | Ar | 1000 | 60 | 1.00E+18 |

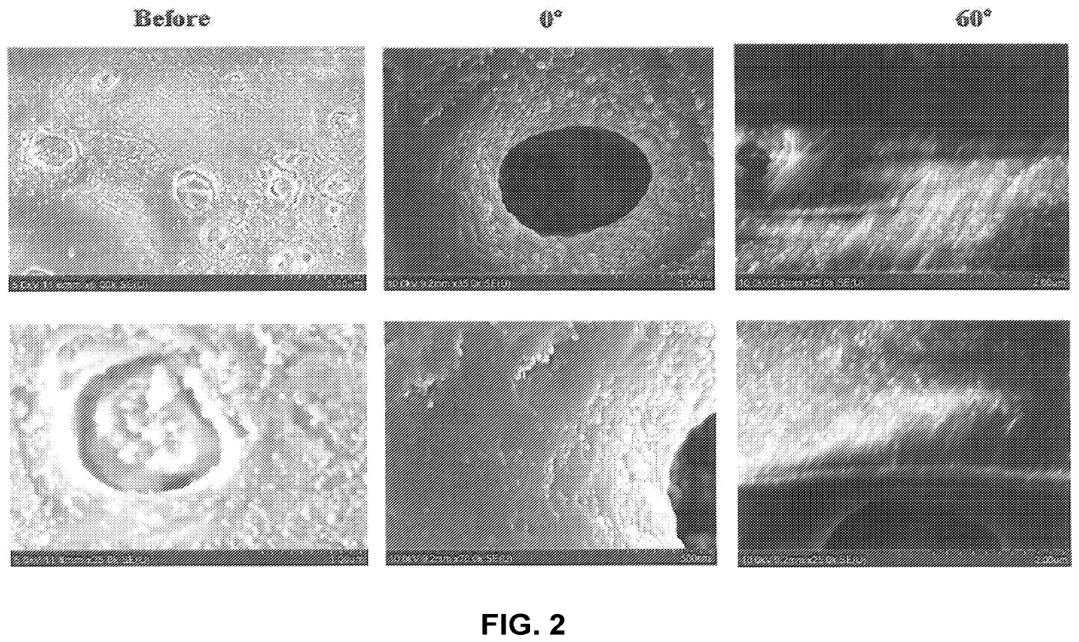

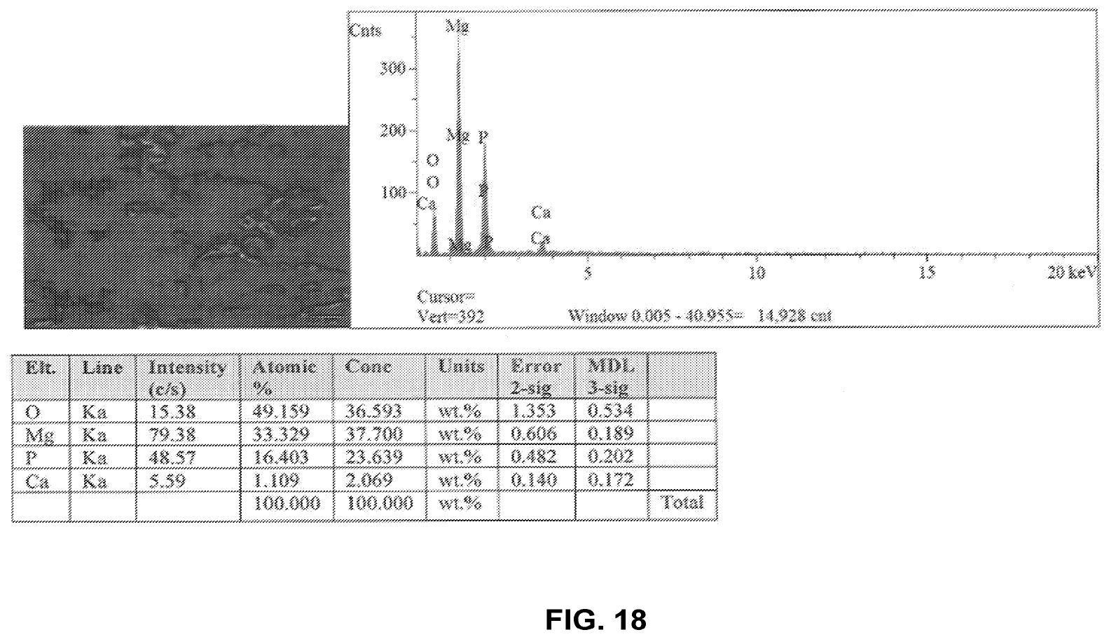

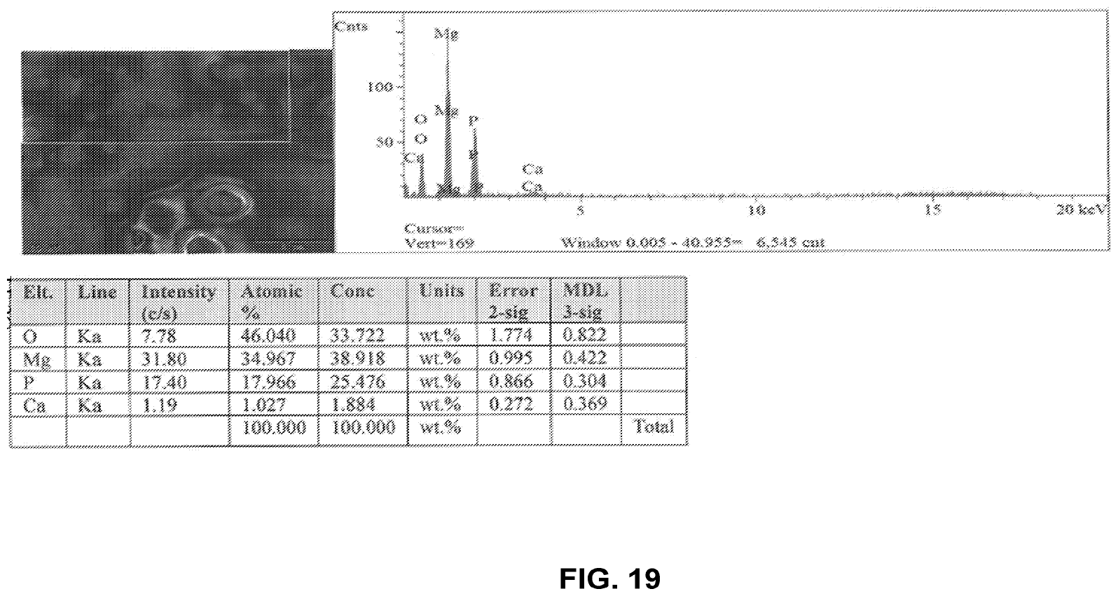

For example, in the Mg_PA_001 and 002 samples two distinct nanostructures are formed. For 0°, we observe 10-50 nm nanoscale nodules with ripples of 25-nm pitch, whereas at 60° nanocones with columns inclined towards the flux direction are seen of size that varies between 200-nm width diameter and length of about 1-2 microns with tips 10-100 nm thin. Controls compared to O₂ exposed anodized Mg samples are shown in FIG. 2. Further EDS analysis shows hydroxyapatite formation on the same samples when immersed in simulated body fluid for 24 hours (see FIGS. 18 and 19).

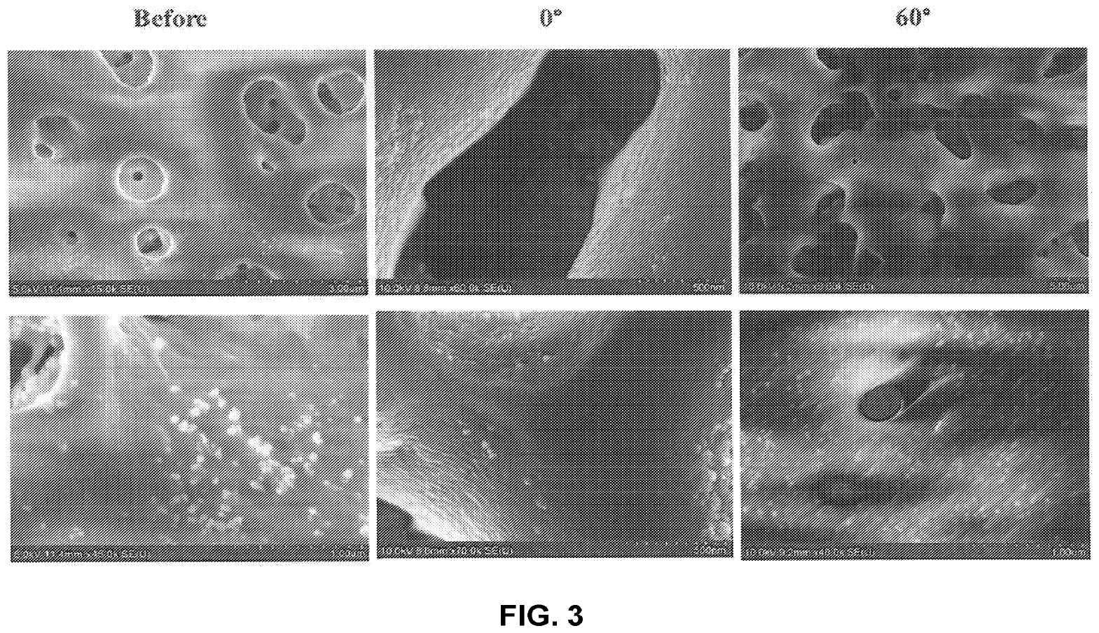

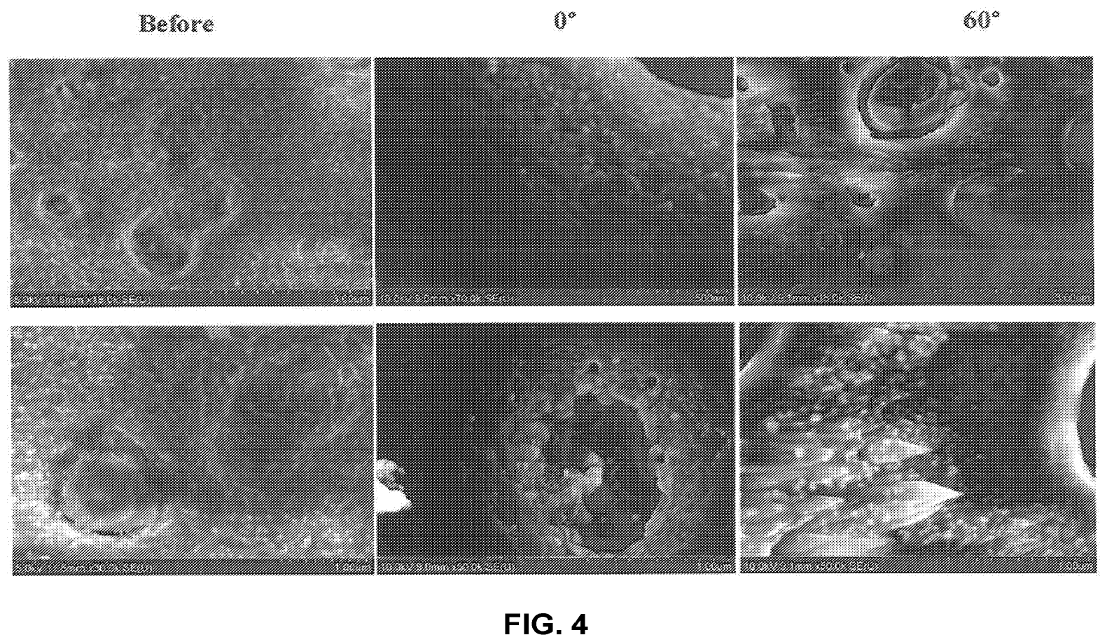

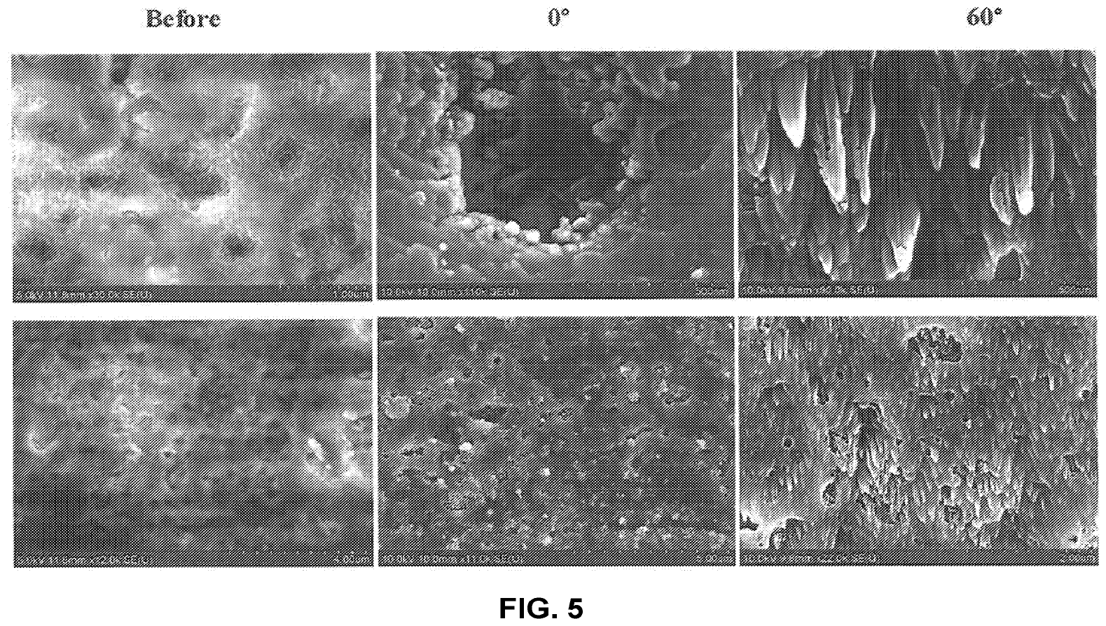

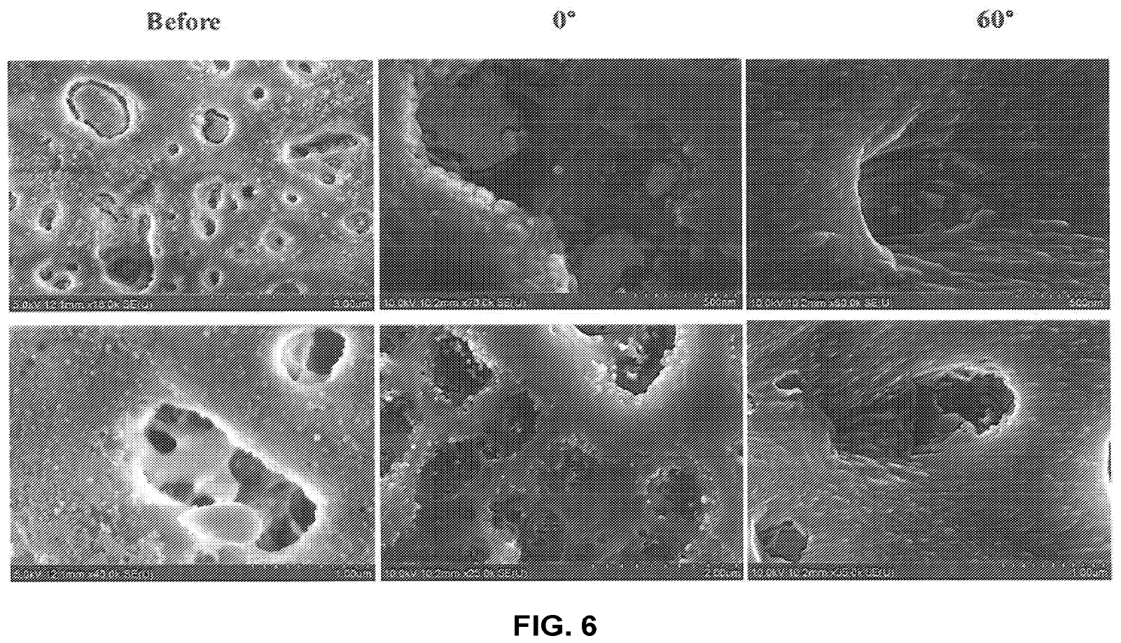

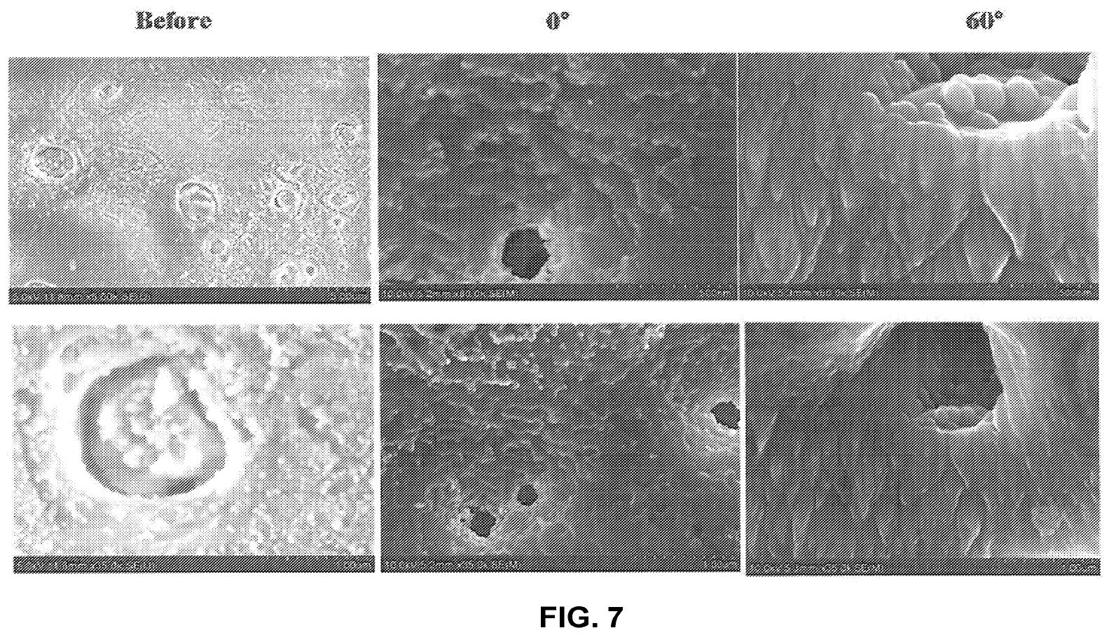

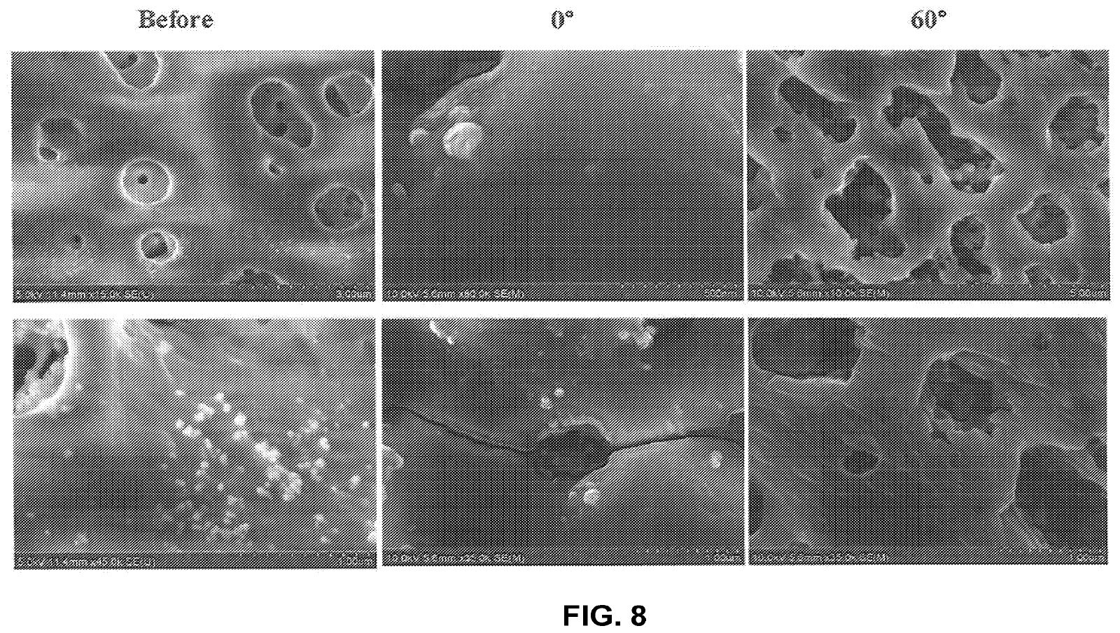

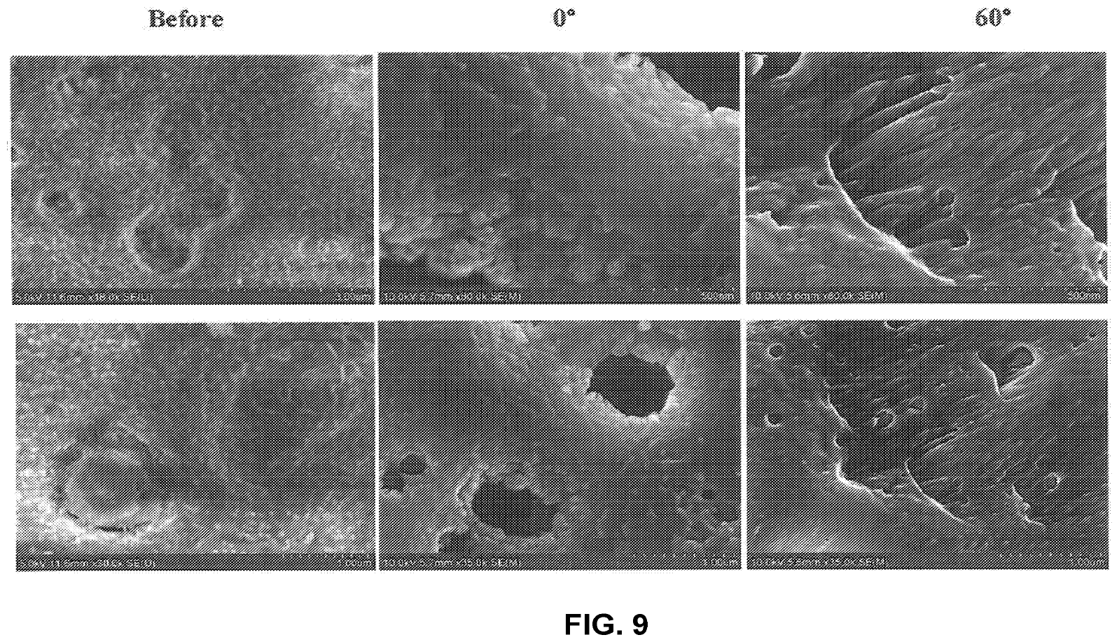

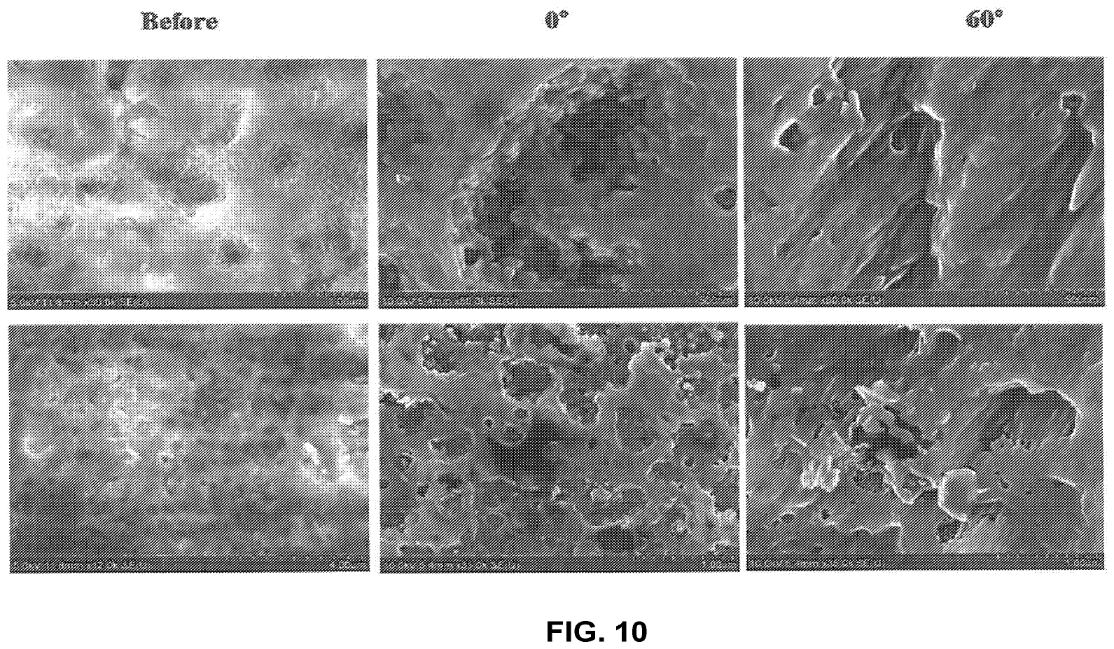

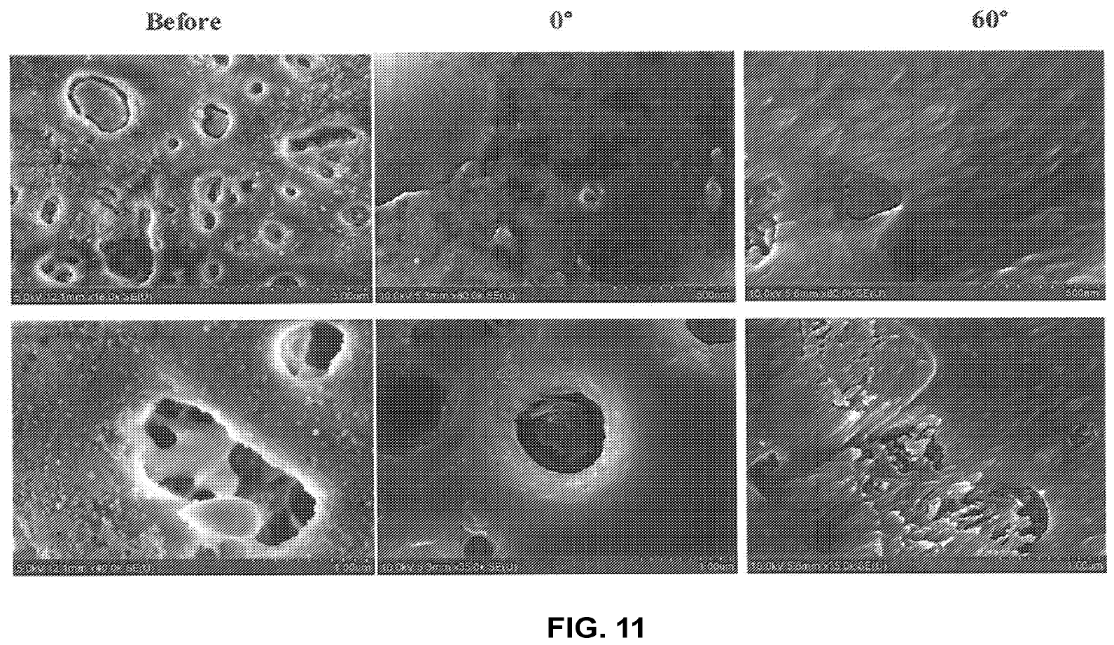

Surface structural modifications and nanostructuring by DPNS of all the anodized samples are presented in FIGS. 2-6 for O₂ ions and FIGS. 7-11 for Ar ions. There are two distinct nanostructures at various angles of incidence for both the gases. For 0°, nanoripples can be seen, whereas at 60° nanocolumns with columns inclined towards the flux direction are seen.

Osseoconductivity and osseointegration properties of DPNS samples may be tested using osteoblast cultures. Osteoblast survival is summarized in Table 3.

TABLE 3
Summary of osteoblast survival cultured
on modified magnesium surfaces.

| Sample | Cell | No cell |
|---|---|---|
| Mg_PA_001 | ✓ ✓ | |
| Mg_PA_002 | | x |
| Mg_PA_003 | ✓ ✓ | |
| Mg_PA_004 | | x |
| Mg_PP_001 | | x |
| Mg_PP_002 | | x |
| Mg_PP_003 | ✓ | |
| Mg_PP_004 | | x |
| Mg_PT_001 | ✓ ✓ ✓ | |
| Mg_PT_002 | | x |
| Mg_PT_003 | ✓ ✓ ✓ | |
| Mg_PT_004 | | x |
| Mg_PFCV_001 | | x |
| Mg_PFCV_002 | ✓ ✓ | |
| Mg_PFCV_003 | | x |
| Mg_PFCV_004 | | x |
| Mg_PFCC_001 | ✓ ✓ ✓ | |
| Mg_PFCC_002 | ✓ | |
| Mg_PFCC_003 | | x |
| Mg_PFCC_004 | | x |

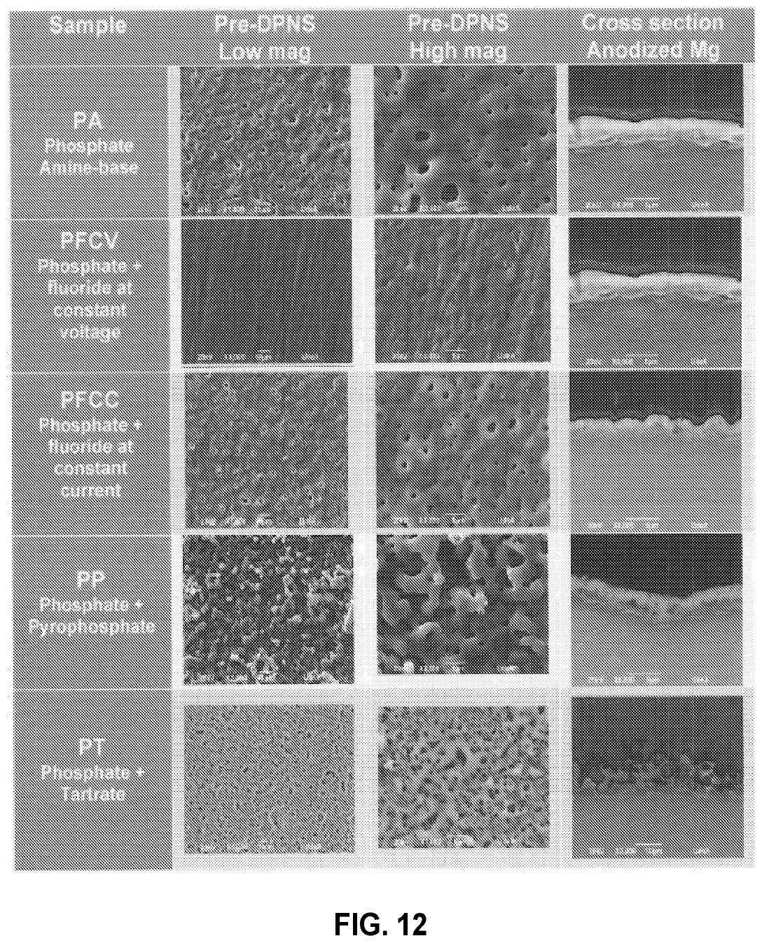

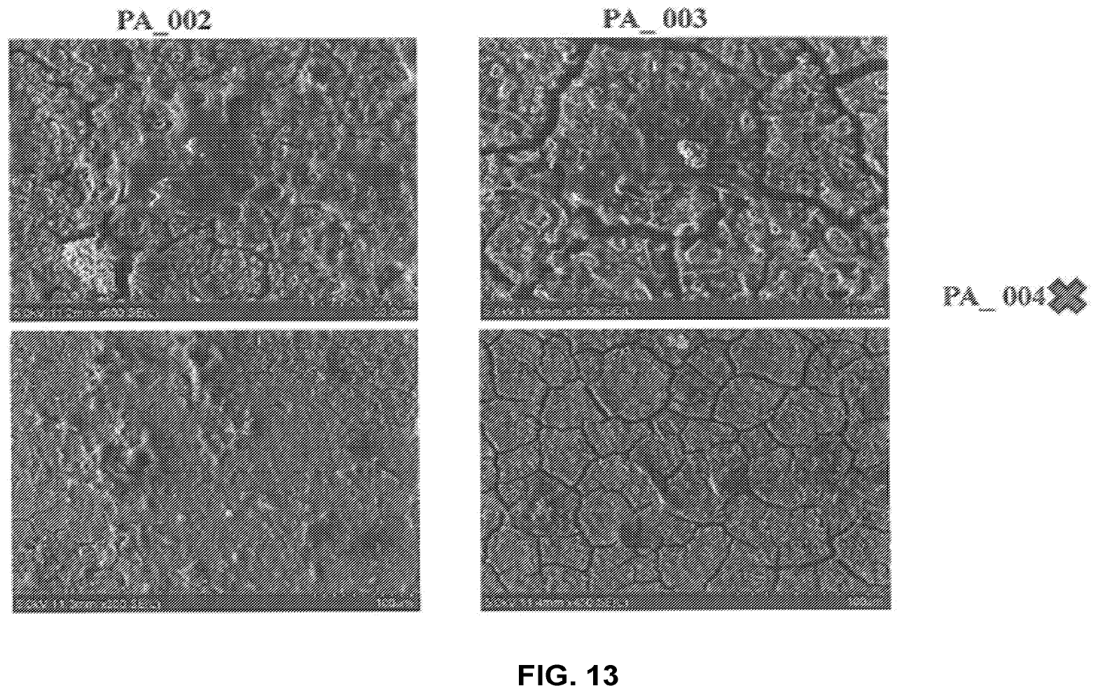

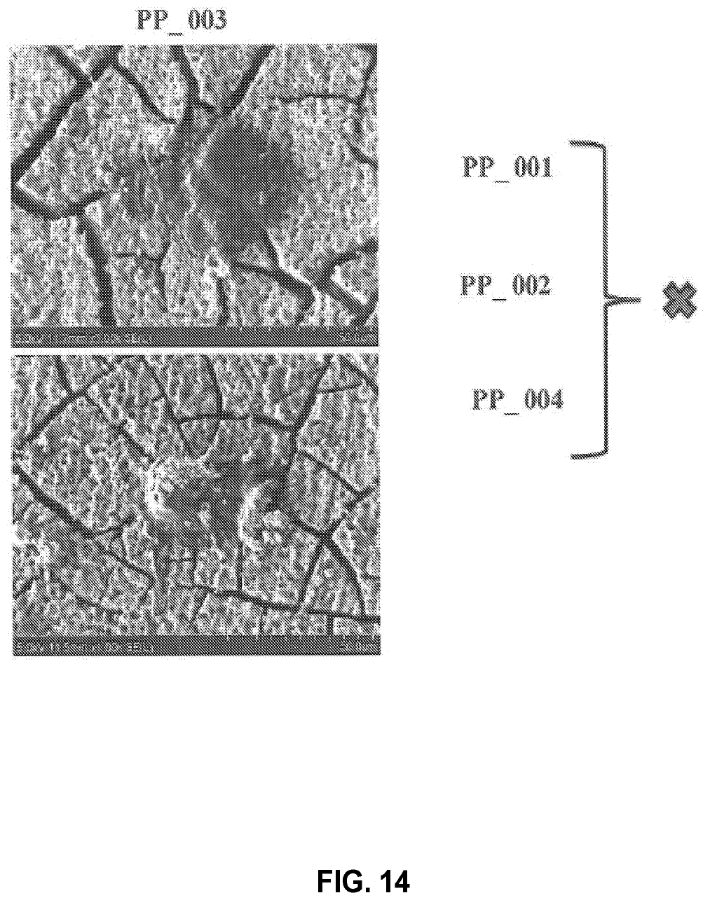

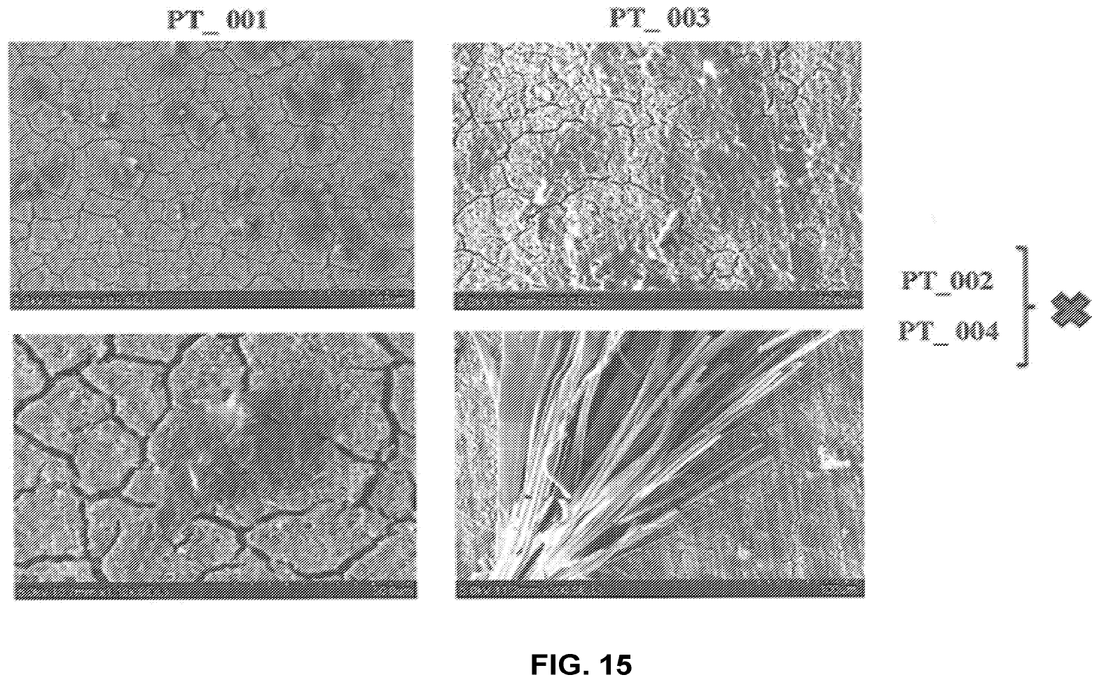

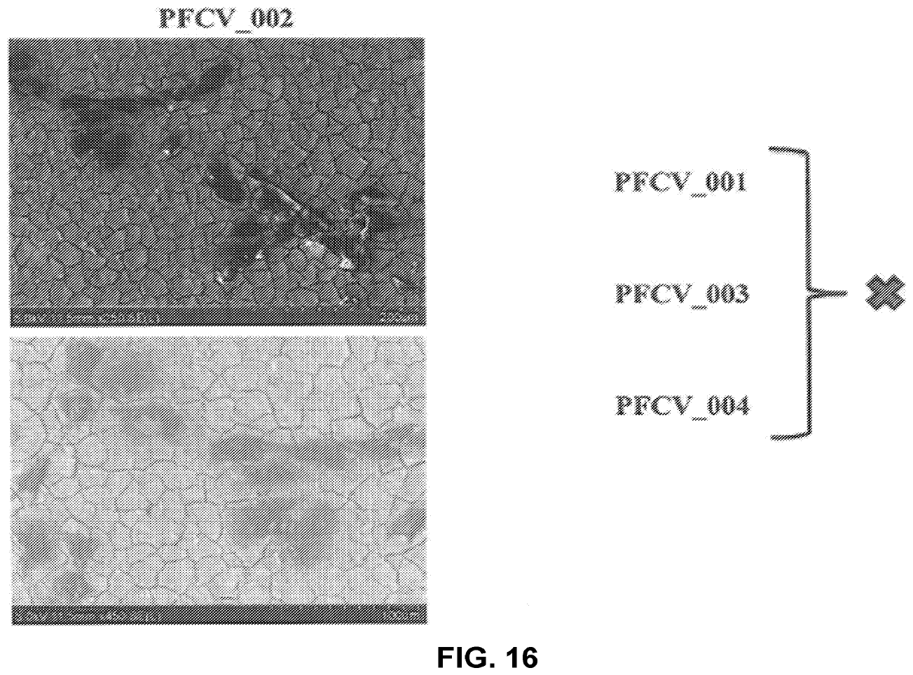

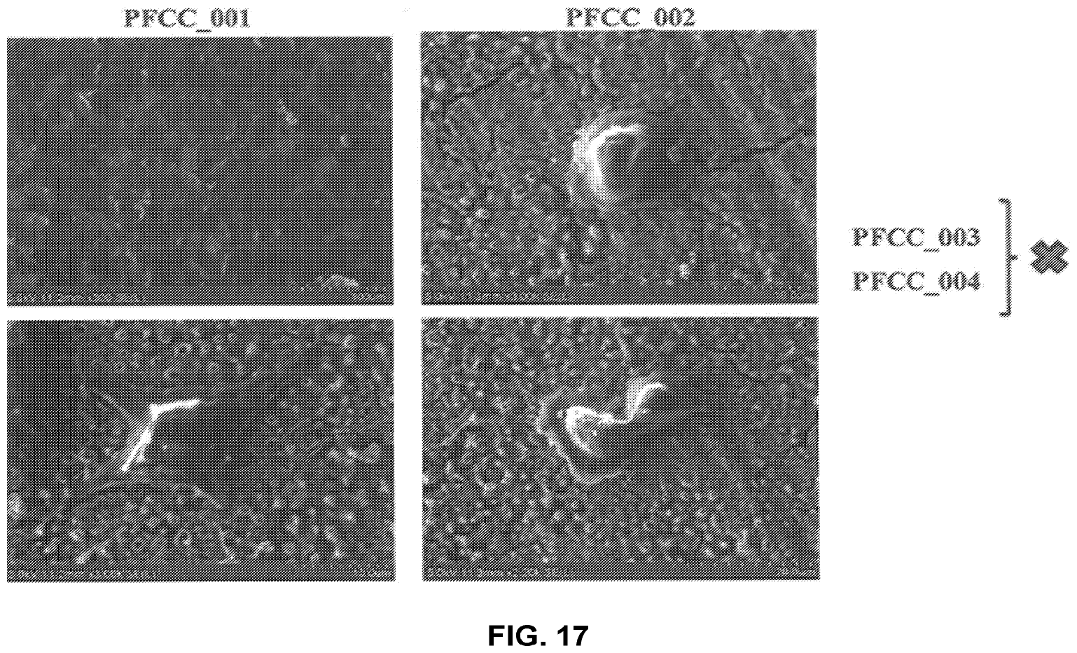

FIG. 12 provides SEM images of pre-DPNS anodized magnesium materials, as described herein. FIGS. 13-17 display osteoblasts cultured onto ion irradiated anodized Mg. The osteoblastic cells exhibited enhanced cell growth for some anodized samples compared to untreated Mg samples. A set of indicators were used to qualify cell growth and proliferation by qualitatively examining cell health and filopodia/lamellapodia development during the culture time on Mg sample surfaces. Table 3 summarizes the results shown in FIGS. 13-17. Each arrow represents a qualitative indicator for the magnitude of improvement in cell growth and proliferation.

The surface properties responding to simulated body fluid (SBF) depend on the surface chemistry, nanotopography, surface charge density, surface free energy controlled by DPNS parameters. For example, a particular nanotopography can drive a particular surface concentration of a component that then drives the Ca/P phases to promote hydroxyapatite formation. A specific surface chemistry can also drive a particular nanotopography on the surface.

Without exposure (e.g. non-surface modified controls) we find no evidence of stable Ca/P phases indicative of HA (hydroxyapatite) formation, which promotes mineralization (e.g. bone formation) and more importantly accelerates this process yielding faster bone growth and increased bone fixation. Due to the sensitive interplay between DPNS controls on substrate surface the substrate conditions provide important mechanisms for HA growth promotion. Anodized magnesium and cellular magnesium substrates (e.g. magnesium sponges and foams) both exhibit Ca/P phase formation by DPNS, but they do so differently.

Hydroxyapatite formation—EDS analysis on samples PA Mg_001 and 002 show hydroxyapatite formation after the samples were immersed in simulated body fluid for 24 hours and are provided in FIGS. 18 and 19.

### Example 2—Preferential Increase of Aluminum Conentration at Modified Surface

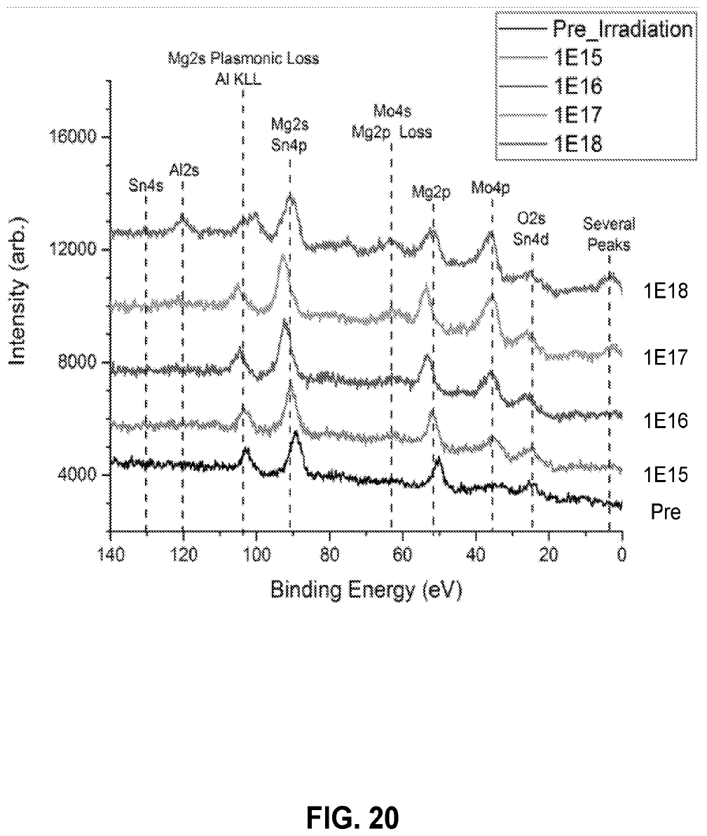

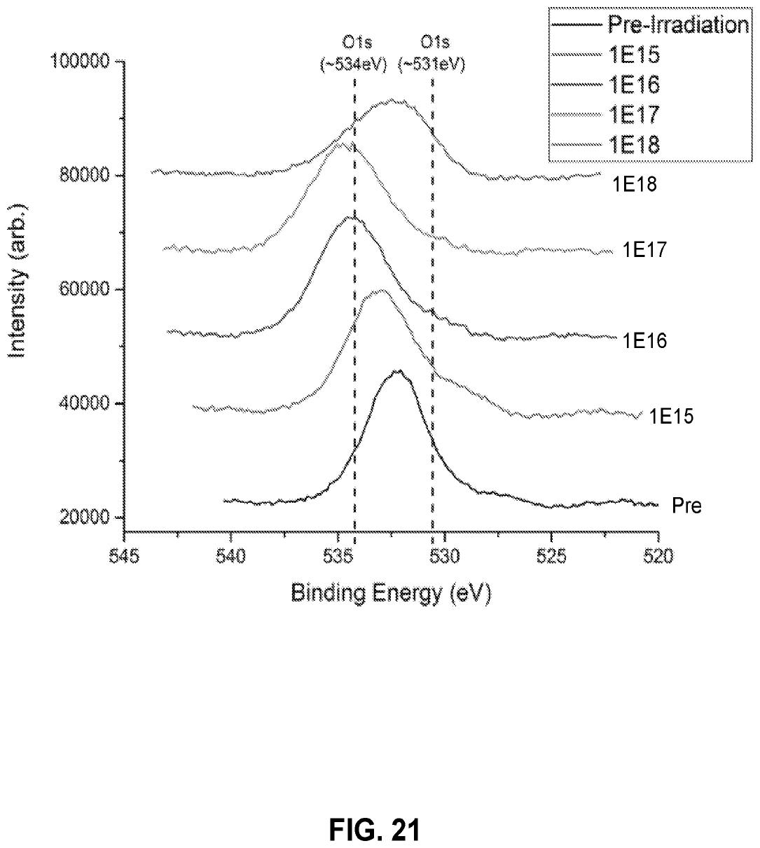

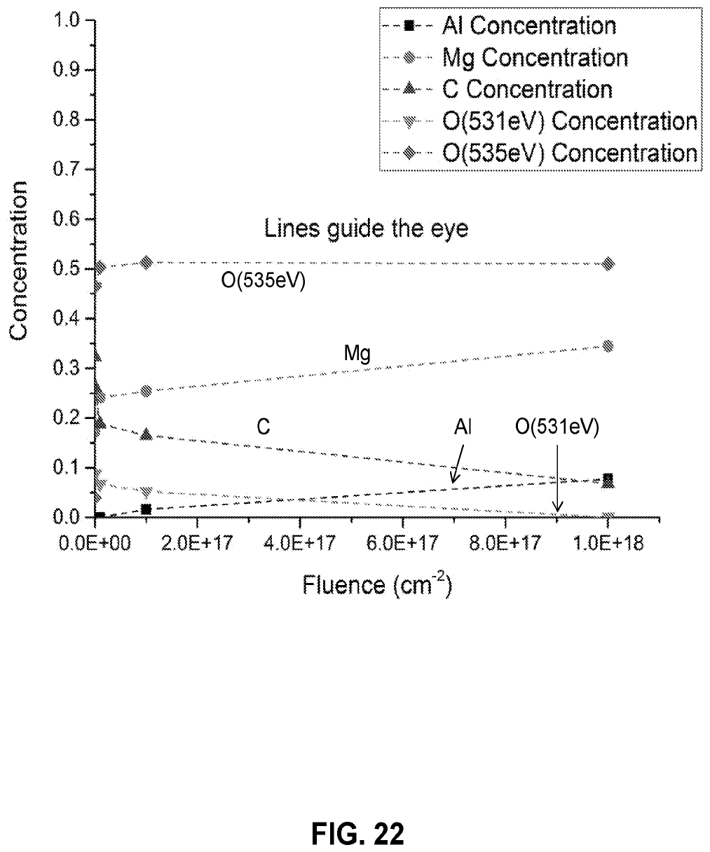

FIGS. 20-22 provide X-ray photoelectron spectroscopy data of a magnesium alloy sample surface treated with DPNS and illustrate the preferential increase of in-situ Al at the surface. The magnesium alloy sample is the Mg AZ 31 composition having 500 micron pores. The composition of Al is driven to about 10% at the modified surface when exposed to a fluence of 1×10¹⁸ particles per second per square centimeter as shown in FIG. 22. Additionally, the concentration of Sn (another component of the alloy) is not appreciably increased. The increase of Al while avoiding additional Sn at the modified surface improves hydroxyapatite formation and allows for precise control of corrosion rate.

FIG. 22 also illustrates that O1s at 531 eV concentration is decreased while O1s at 535 eV remains stable. This indicates that the MgO at the surface being modified is not decreasing while the Al is increasing. The data also suggests that Carbon decreases as impurities such as CO are removed from the modified surface.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A magnesium composition comprising:
a magnesium containing substrate having a surface; wherein said surface has a plurality of nanoscale domains characterized by a surface geometry providing a selected multifunctional bioactivity;
wherein each of said nanoscale domains has at least one lateral spatial dimension selected over the range of 3 nm to 1 [symbol] m and a vertical spatial dimension less than 500 nm.

2. A magnesium composition comprising:
a magnesium containing substrate having a surface; wherein said surface has a plurality of nanoscale domains characterized by a surface geometry providing a selected multifunctional bioactivity;
wherein said nanoscale domains are generated by exposing said surface to one or more directed energetic particle beam characterized by one or more beam properties.

3. The composition of claim 1 or 2, wherein said magnesium containing substrate is a magnesium metal, a magnesium alloy, an anodized magnesium metal, an anodized magnesium alloy or a magnesium oxide.

4. The composition of claim 1 or 2, wherein said selected multifunctional bioactivity is with respect to an in vivo or in vitro activity relative to an unmodified magnesium containing substrate.

5. The composition of claim 4, wherein said in vivo or in vitro activity is a change in rate of bioresorption.

6. The composition of claim 5, wherein said change in rate of bioresorption is selected from the range of 0.10 mm/year to 0.18 mm/year.

7. The composition of claim 4, wherein said in vivo or in vitro activity is a decrease in hydrogen generation.

8. The composition of claim 7, wherein said decrease in hydrogen generation is greater than or equal to 0.6 ml/cm².

9. The composition of claim 4, wherein said in vivo or in vitro activity is an enhancement in bioresorption, hydrogen generation, cell adhesion activity, cell shape activity, cell proliferation activity, cell migration activity, cell differentiation activity, anti-bacterial activity, bactericidal activity, anti-inflammatory activity, osseointegration activity, biocorrosion activity, cell differentiation activity, immuno-modulating activity during acute or chronic inflammation or any combination of these.

10. The composition of claim 9, wherein said enhancement of in vivo or in vitro activity is equal to or greater than 100%.

11. The composition of claim 1 or 2, wherein said magnesium containing substrate is anodized.

12. The composition of claim 1 or 2, wherein said nanoscale domains comprise calcium phosphate.

13. The composition of claim 1 or 2, wherein said nanoscale domains comprise an increase in Al₂O₃ content relative to the Al₂O₃ content of other regions of said magnesium containing substrate no having said nanoscale domains.

14. The composition of claim 1 or 2, wherein said surface geometry is spatial distribution of relief features, recessed features, localized regions characterized by a selected composition, phase, crystallographic texture, or any combination of these.

15. The composition of claim 1 or 2, wherein said surface geometry is a periodic or semi-periodic spatial distribution of said nanoscale domains.

16. The composition of claim 1 or 2, wherein said surface geometry is a selected topology, topography, morphology, texture or any combination of these.

17. The composition of claim 1 or 2, wherein each of said nanoscale domains are characterized by a vertical spatial dimension of less than or equal to 50 nm.

18. The composition of claim 1 or 2, wherein each of said nanoscale domains are characterized by a vertical spatial dimension selected over the range of 10 nm to 250 nm.

19. The composition of claim 1 or 2, wherein said nanoscale domains comprise nanowalls, nanorods, nanoplates, nanoripples or any combination thereof having lateral spatial dimensions selected over the range of 10 to 1000 nm and vertical spatial dimensions of less than or equal to 250 nm.

20. The composition of claim 19, wherein said nanowalls, nanorods, nanoplates or nanoripples are inclined towards a direction oriented along a selected axis relative to said surface.

21. The composition of claim 19, wherein said nanowalls, nanorods, nanoplates or nanoripples are separated from one another by a distance of less than or equal to 100 nm.

22. The composition of claim 1 or 2, wherein said nanoscale domains comprise discrete crystallographic domains.

23. The composition of claim 1 or 2, wherein said nanoscale domains characterized by a chemical composition different from the bulk phase of said magnesium containing substrate.

24. The composition of claim 1 or 2, wherein said surface geometry provides an enhancement in vivo or in vitro activity with respect to cell adhesion proliferation activity and migration greater than or equal to 100%.

25. The composition of claim 1 or 2, wherein said surface geometry provides an enhancement in vivo or in vitro activity with respect to anti-bacterial activity and bactericidal activity greater than or equal to 100%.

26. The composition of claim 1 or 2, wherein said surface geometry provides a local in vivo increase in pH, wherein said pH is increased by 0.5 or more.

27. The composition of claim 1 or 2, wherein said surface geometry provides an enhancement of a selected physical property of said substrate.

28. The composition of claim 27, wherein said physical property is hydrophilicity, hydrophobicity, surface free energy, surface charge density or any combination of these.

29. The composition of claim 27, wherein said enhancement of selected physical property is equal to or greater than 25%.

30. The composition of claim 1 or 2, wherein said magnesium containing substrate is a biocompatible substrate.

31. The composition of claim 2, wherein the directed energetic particle beam is a broad beam, focused beam, asymmetric beam, reactive beam or any combination of these.

32. The composition of claim 2, wherein said one or more beam properties is intensity, fluence, energy, flux, incident angle, ion composition, neutral composition, ion to neutral ratio or any combinations thereof.

33. The method of claim 1 or 2, wherein said magnesium containing substrate is a mesoporous, microporous or nanoporous substrate.

34. A method of fabricating a bioactive magnesium composition comprising:
providing a magnesium containing substrate having a substrate surface; and
directing a directed energetic particle beam onto said substrate surface, thereby generating a plurality of nanoscale domains on said surface;
wherein said directed energetic particle beam has one or more beam properties selected to generate said plurality of nanoscale domains characterized by a surface geometry providing a selected multifunctional bioactivity.

35. The method of claim 34, wherein the directed energetic particle beam is a broad beam, focused beam asymmetric beam or any combination of these.

36. The method of claim 34, wherein said step of directing said directed energetic particle beam onto said substrate surface comprises directed plasma nanosynthesis (DPNS), Direct Seeded Plasma Nanosynthesis (DSDPNS), Direct Soft Plasma Nanosythesis (DSPNS) or any combination of these.

37. The method of claim 34, wherein said one or more beam properties is intensity, fluence, energy, flux, incident angle, ion composition, neutral composition ion to neutral ratio or any combinations thereof.

38. The method of claim 34, wherein said directed energetic particle beam comprises one or more ions, neutrals or combinations thereof.

39. The method of claim 38, wherein said ions are Ne ions, Kr ions, Ar ions, Xe ions, N ions or a combination thereof.

40. The method of claim 38, wherein said directed energetic particle beam is generated from an energetic 02 precursor.

41. The method of claim 34, wherein said directed energetic particle beam anodizes said magnesium containing substrate thereby generating an anodized bioactive magnesium substrate.

42. The method of claim 34, wherein said one or more beam properties comprise incident angle and said incident angle is selected from the range of 0° to 80°.

43. The method of claim 34, wherein said one or more beam properties comprise fluence and said fluence is selected from the range of 1×10¹⁶ cm⁻² to 1×10²⁰ cm⁻².

44. The method of claim 34, wherein said one or more beam properties comprise energy and said energy is selected from the range of 0.05 keV to 10 keV.

45. The method of claim 34, wherein said multifunctional bioactivity comprises bioresorption.

46. A method of fabricating a bioactive magnesium substrate comprising:
providing said magnesium containing substrate having a substrate surface; and
directing a first directed energetic particle beam and a second directed energy particle beam onto said substrate surface, thereby generating a plurality of nanoscale domains on said surface;
wherein said first directed energetic particle beam has one or more first beam properties and said second directed energetic particle beam has one or more second beam properties; and
wherein at least one of said first beam properties is different than at least one of said second beam properties and said first beam properties and said second beam properties are independently selected to generate said plurality of nanoscale domains characterized by a surface geometry providing a selected multifunctional bioactivity.
